(19) United States
(12) Patent Application Publication
(10) Pub. No.: US 2025/0002597 A1
(43) Pub. Date: Jan. 2, 2025

(54) METHOD FOR OPTIMIZING DRUG SELECTION

(71) Applicant: Edward Lichten, Wellington, FL (US)

(72) Inventor: Edward Lichten, Wellington, FL (US)

(21) Appl. No.: 18/745,376

(22) Filed: Jun. 17, 2024

Related U.S. Application Data
(60) Provisional application No. 63/524,055, filed on Jun. 29, 2023.

(51) Int. Cl. C07K 16/28; A61K 31/568; A61K 31/58; A61K 31/585; A61K 39/395; G01N 33/68

(52) U.S. Cl. CPC C07K 16/2875; A61K 31/568; A61K 31/58; A61K 31/585; A61K 39/3955; G01N 33/6863; C07K 2317/24; G01N 2333/525; G01N 2500/02

(57) ABSTRACT

A method for determining drug administration to reduce or block the activity of pro-inflammatory cytokines by determining the efficacy of pain reducing drugs and the enhancement thereof by blocking the signal for cytokine release and blocking estrogen receptor beta.

### CROSS-REFERENCE TO RELATED APPLICATIONS

This Application is a Completion Application of co-pending United States Provisional Patent Application, 63/524,055 filed on Jun. 29, 2023, for “Method for Optimizing Drug Selection,” the disclosure of which is hereby incorporated by reference.

### PRIOR ART

As is known to those skilled in the art to which the present invention pertains, pro-inflammatory cytokines act to make disease worse, whereas others serve to reduce inflammation and promote healing. These are anti-inflammatory. Those cytokines which make disease worse are denominated as pro-inflammatory.

Cytokines, per se, are regulators of host responses to infection, immune responses, inflammation and trauma. Attention has focused on blocking cytokines which are harmful to the host, particularly during overwhelming infection.

Interleukin (IL-1) and tumor necrosis factor (TNF) are pro-inflammatory cytokines and when they are administered to humans, they produce fever, inflammation, tissue destruction and in some cases, shock and death. Reducing the biological activities of IL-1 and TNF, to date, has been accomplished by using specific strategies which involve neutralizing antibodies, estrogen alpha and beta receptors, receptor antagonists and inhibitors of proteases that convert inactive precursors to active, mature molecules. The blocking of IL-1 or TNF has been highly successful in patients with rheumatoid arthritis, inflammatory bowel disease, etc.

Anti-inflammatory cytokine therapy which prevents cytokine storms or cytokine release syndrome (CRS) is desirable. However, it remains difficult to identify those patients who would benefit from anti-inflammatory cytokine therapy for various inflammatory conditions, and, necessarily, enhancement of pain relief.

The present invention as described hereinbelow provides a determinant for drug administration to prevent CRS and thereby enhance pain relief.

### BACKGROUND OF THE INVENTION

The present invention concerns means and methods for determining and enhancing the efficacy of pain releasing drugs. More particularly, the present invention concerns treatments for increasing pain relief for post-surgical patients.

### SUMMARY OF THE INVENTION

The present invention provides in a first aspect, a method for determining drug administration to reduce or block the activity of pro-inflammatory cytokines.

In another aspect hereof, it is well known that in many surgical procedures, there is pain associated with recovery which can be enduring. The present invention further enables the determination of the efficacy of the pain reducing drug and the enhancement thereof.

### DETAILED DESCRIPTION OF THE INVENTION

It is well documented that pro-inflammatory cytokines such as IL-1 β IL-6 and TNF are involved in the process of pathological pain. By determining which drug, alone or in combination with other drugs, including steroidal compounds, reduces the pro-inflammatory cytokines, pain, per se is reduced or obviated.

As is known to those skilled in the art to which the present invention pertains, estrogen receptor beta is a nuclear receptor which is activated by estrogen.

It is postulated that normalizing the estrogen receptor beta will stop the signal for pro-inflammatory cytokine release which, in turn, will prevent or inhibit CRS.

As noted, the present method provides a determinant for the medical treatment or drug necessary to preclude or inhibit the release of the pro-inflammatory cytokines. As noted, it is believed that by blocking the estrogen receptor beta or normalizing the estrogen receptor beta, this will stop the signal for pro-inflammatory cytokine release.

In accordance with the present invention, in a first aspect hereof, to determine the efficacy of drugs to prevent or inhibit CRS, a blood sample is divided into multiple fractions. The fractions are then treated or exposed to one or more drugs and measured for inflammatory cytokines. Where there is a reduction in the cytokines, it is an indicator that the drug is doing its intended reduction of inflammation.

More particularly and in accordance with the present invention, in practicing the present method, a blood sample drawn from a patient is subjected to an ELISA analysis (enzyme-linked immunosorbent assay) to analyze eight different pro-inflammatory cytokines to determine which selected drug reduces the pro-inflammatory cytokines to the greatest degree. That drug or a combination thereof defines the recommended treatment for inflammation and for increasing pain relief.

In practicing the present invention, the blood sample is drawn and is then centrifuged to separate out the blood serum from the protoplasm. This is done by taking the blood sample and putting it into a heparin tube, centrifuging it, which then separates out the T-lymphocytes, i.e., blood serum from red cells. The T-lymphocytes are further separated out from the serum and cultured and then placed in a platelet and subjected to the ELISA treatment. It is the recovered T-lymphocytes that have the pro-inflammatory cytokines.

Thereafter, the T-lymphocytes are admixed with the pro-inflammatory cytokine serums. The anti-serum is used for each one of the pro-inflammatory cytokines. The type of cytokine will determine the anti-serum used.

By implementing this procedure, the methodology will normalize the estrogen receptor beta to stop the signal for pro-inflammatory cytokine release.

Table 1 below is a specific example of the implementation of the present method.

In practicing the present invention, an in-vitro laboratory using standard ELISA test kits offers quantitative measurements of estrogen receptors alpha and beta, and the selected interleukins are used. The human TNF-α ELISA (Enzyme-Linked Immunosorbent Assay) is incorporated for the quantitative measurement of Human TNF-α in the serum, plasma, cell culture supernatants and urine. A lymphocyte toxicity assay or LTA is then used to measure and determine toxicity and whether the cells show any effect of the various drug therapies in reducing or increasing proliferation and propagation of interleukins.

TABLE 1

| | | | | | | |
|---|---|---|---|---|---|---|
| IL-1β | IL-2 | IL-6 | IL-8 | TNFα | VEGF | Toxicity assay |
| | | | | | | (neg) |

The present invention is particularly effective in determining the efficacy of the drug alone by measuring the lymphocyte toxicity assay and then combining the prescribed drug with various anabolic steroidal compounds.

Exemplifying the present invention is Table 2 below which sets forth a lymphocyte toxicity assay for a post-mastectomy treatment for reducing pain by measuring the efficacy of combining a known drug, Inflectra, with a steroidal compound which is an anti-tumor necrosis alpha inhibitor as the base.

As is apparent from Table 2 below, the combination of testosterone, nandrolone and Inflectra had the highest viability of the cells and was effective in precluding cytokine release.

TABLE 2
Lymphocyte toxicity assay

| Therapeutic | Viability % | Toxicity | Combination of | Therapeutics | Viability % |
|---|---|---|---|---|---|
| Cels- Control | 100 | | | | |
| INFLECTRA | 160 | | Testosterone +Nandrolone + INFLECTRA | | 175% |
| Testosterone | 150 | | INFLECTRA | Testosterone | 150% |
| Nandrolone | 158 | | INFLECTRA | Nandrolone | 168% |
| Stanozolol | | 14% | INFLECTRA | Stanozolol | 105% |
| Oxandrolone | 117 | | INFLECTRA | Oxandrolone | 160% |

Interpretation
Patient/blood sample arrived in good condition. Cell viability after growing up in a media was considered
100%. The same amount of cells grew in the same condition, have been exposed with the therapeutics:
Inflectra (anti-tumor necrosis alpha inhibitor), Testosterone, Nandrolone, Stanozolol, Oxandrolone.
The only medication that was toxic to the cell was Stanozolol. However, the combination of Stanozolol
and Inflectra was not toxic to the cells.
The best combination leading to the highest viability of the cells was Testosterone + Nandrolone +
INFLECTRA
Inflammatory markers in serum
Vascular endothellal factor (VEGF) −175 pg/mL Normal values 30-90 pg/mL
Metallo-proteinase C (MMP-3) 4.35 ng/mL Normal value 1.5-2.5 ng/mL
Tumor necrosis factor alpha (TNF) −125 pg/mL Normal values 20-70 pg/mL
Tumor necrosis factor alpha (TNF) receptor 2 −95 pg/mL Normal values 20-70 pg/mL
Interpretation
The inflammatory markers are higher in comparison with the normal values. Per our experience are
compatible with moderate UC.
Standard curve to all the readings R-0.90.
Method: ELISA
Kits: Human VEGF, Human TNF, Human TNF receptor II (In Vitrogen, Thermo-Fisher Scientific)-
Analytical sensitivity 7.9 pg/mL;' Assay range 15.6-1,000 pg//mL
Human MPP kit (Biogen) -Analytical sensitivity −0.5 pg/mL

In subsequent testing with other inhibitors including Humera, the same results were obtained. Thus, by administering the combination set forth above, the pro-inflammatory cytokines and CRS are minimized.

It is believed that this procedure is appliable to various autoimmune diseases as well as other organic issues, including PTSD, Coronavirus, breast cancer, Crohn's disease, IBD, IBS, etc.

1. A method for precluding or inhibiting a pro-inflammatory cytokine storm in a patient, comprising:
blocking or normalizing the estrogen receptor beta in the blood of a patient, whereby the signal for pro-inflammatory cytokine release is blocked by administering a drug for preventing the signal for causing the release.

2. The method of claim 1 wherein the drug for blocking the signal is determined by:
a) obtaining a blood sample of a patient;
b) conducting an ELISA analysis of the sample;
c) analyzing eight different pro-inflammatory cytokines
d) exposing the pro-inflammatory cytokines to at least one selected drug to determine which of the at least one selected drug reduces the cytokines to the greatest degree and, thereafter, administering the at least one selected drug to the patient to reduce cytokine release.

3. A method for reducing pain in a patient by blocking the estrogen receptor beta, which comprises:
following the protocol of claim 2.

4. The method of claim 3 wherein the at least one drug is selected from the group consisting of Inflectra and Inflectra with an anabolic steroid.

5. The method of claim 4 wherein the steroid is selected from the group consisting of:
testosterone, nandrolone, stanozolol and oxandrolone.

6. The method of claim 5 wherein the drug is an admixture of Inflectra, testosterone and nandrolone.
